(19) United States
(12) Patent Application Publication
(10) Pub. No.: US 2011/0270397 A1
(43) Pub. Date: Nov. 3, 2011

(54) SPINAL COVERING DEVICE

(76) Inventor: Jean-Marc Mac-Thiong, Montreal (CA)

(21) Appl. No.: 13/143,371

(22) PCT Filed: Jan. 6, 2010
(86) PCT No.: PCT/CA2010/000007

§ 371 Date: Jul. 6, 2011

Related U.S. Application Data
(60) Provisional application No. 61/193,894, filed on Jan. 6, 2009.

(51) Int. Cl. A61F 2/44

(52) U.S. Cl. 623/17.11

(57) ABSTRACT

A spinal covering device for covering exposed neural spinal elements after a spinal procedure includes a body at least substantially or completely free of openings and first and second support planes extending longitudinally along the body adapted to overlay or abut remaining tissues on opposite sides of the exposed neural spinal elements such that the device forms a spinal canal portion protecting the neural spinal elements. The device has an inner surface at least partially defining an inner surface of the spinal canal portion and an outer surface opposite of the inner surface, at least the outer surface allowing bone graft deposition or bone fusion thereon.

### CROSS-REFERENCE TO RELATED APPLICATIONS

The present application claims priority on U.S. Provisional Patent Application No. 61/193,894 filed Jan. 6, 2009, the entire contents of which is incorporated herein by reference.

### FIELD OF THE INVENTION

The present invention relates to a spinal covering device and to a method for protecting neural spinal elements following a spinal procedure.

### BACKGROUND ART

Neural spinal elements can be exposed after various spinal procedures such as laminectomy, spinal decompression, spinal release, osteotomy and discectomy. Increase in the space available for neural elements can also be required in case of spinal stenosis. When a fusion procedure is performed, placing bone graft on exposed neural elements can cause undue neural compression. The surface available for fusion is reduced since the bone graft is laid only on the remaining bone surfaces, so that bone healing can be unsuccessful or inadequate. Bone graft applied on the remaining bone surfaces can also be displaced and can potentially injure the spinal neural elements. In addition, scarring and bone regrowth can later compress the exposed neural elements and as such necessitate additional revision surgery. The presence of scar and/or bone regrowth over the neural elements can significantly increase the risk of complications and injury to the neural elements during revision surgery.

Even when no fusion procedure is performed, scarring and bone regrowth can later compress the exposed neural elements and cause major symptoms to the patient or necessitate the need for additional surgery to decompress the neural elements. Some surgeons have applied biological non-rigid material to reduce scar formation but such a procedure generally does not prevent mechanical compression by overlying soft or bony tissues. Other surgeons have used laminoplasty procedures to expand the spinal canal using the patient's own vertebral posterior arch that is displaced and stabilized, in an attempt to prevent scar formation in addition to provide greater space for neural elements. However, the coverage and space available for neural elements are limited by the patient's bone morphology. These procedures also usually leave an uncovered area with exposed neural elements on the side of bone cut, which disrupts the continuity of the posterior arch. Also, they are usually technically difficult to perform and can lead to complications such as fractures and failure of fixation, and the extent of final coverage and decompression is generally difficult to predict or control with precision.

Accordingly, improvements are desirable.

### SUMMARY OF INVENTION

It is an object of the present invention to provide an improved spinal covering device and method of protecting exposed neural spinal elements.

In accordance with the present invention, there is provided a spinal covering device for covering exposed neural spinal elements after a spinal procedure, the device comprising a body at least substantially free of openings and extending longitudinally from a first end to a second end, and first and second support planes extending longitudinally along the body, the first and second support planes being adapted to overlay or abut remaining tissues on opposite sides of the exposed neural spinal elements such that the device forms a spinal canal portion protecting the neural spinal elements, the device having an inner surface at least partially defining an inner surface of the spinal canal portion and an outer surface opposite of the inner surface, at least the outer surface allowing bone graft deposition or bone fusion thereon.

Also in accordance with the present invention, there is provided a spinal covering device for covering at least one exposed neural spinal element after a spinal procedure comprising an arcuate shaped body at least substantially free of openings, the arcuate shape being defined from a first longitudinal side to a second longitudinal side of the body, and first and second support planes respectively extending from the first and second longitudinal sides, the first and second support planes being adapted to overlay or abut remaining vertebral bones such that the device extends over and spaced apart from the exposed neural spinal elements to form a spinal canal portion, the device having an inner surface at least partially defining an inner surface of the spinal canal portion and an outer surface opposite of the inner surface, at least the outer surface including a bone extender, enhancer or substitute that has osteoconductive, osteoinductive or osteogenic properties.

Still in accordance with the present invention, there is provided a method for protecting at least one exposed neural spinal element following a spinal procedure, comprising positioning a spinal covering device on remaining soft or bony tissues on each side of the at least one exposed neural spinal element or in abutment with the remaining bony tissues to extend over and spaced apart from the at least one exposed neural spinal element, and contacting bone graft or bone substitute material added over or included in an outer surface of the spinal covering device with the remaining bony tissues to allow the bone graft or bone substitute material to fuse with the remaining bony tissues.

### BRIEF DESCRIPTION OF THE DRAWINGS

Reference will now be made to the accompanying drawings, showing by way of illustration a particular embodiment of the present invention and in which:

FIG. 1a is a perspective view of a spinal covering device in accordance with a first embodiment of the present invention;

FIG. 1b is a perspective view of a spinal covering device in accordance with another embodiment of the present invention;

FIG. 2a is a top view of a portion of a spinal column with a spinal covering device such as shown in FIG. 1a attached to a remaining vertebral bone;

FIG. 2b is a top view of a portion of a spinal column with a spinal covering device such as shown in FIG. 1b attached to the remaining vertebral bone;

FIG. 3 is a top view of a portion of a spinal column with a spinal covering device such as shown in FIG. 1b attached to spinal instrumentation used to stabilize adjacent vertebrae;

FIG. 4 is a side view of a portion of a spinal column with a spinal covering device such as shown in FIG. 1b attached thereto;

FIG. 5 is a top view of a portion of a spinal column illustrating the reattachment of paraspinal muscles on a spinal covering device such as shown in FIG. 1a;

FIG. 6 is a top view of a portion of a spinal column showing a spinal covering device such as shown in FIG. 1a attached to adjacent vertebrae using non-rigid connections;

FIG. 7 is a top view of multiple spinal covering devices such as shown in FIG. 1b placed on a portion of a spinal column;

FIG. 8 is a top view of a multiple-level spinal covering device in accordance with another embodiment of the present invention wherein the device covers the neural elements on multiple vertebral segments;

FIG. 9 is a cross-sectional view of a spinal covering device in accordance with another embodiment of the present invention wherein the device covers the neural elements after removal of the anterior vertebral body;

FIG. 10a is a perspective view of a spinal covering device in accordance with another embodiment of the present invention;

FIG. 10b is a perspective view of a spinal covering device in accordance with another embodiment of the present invention;

FIG. 11a is a perspective view of a spinal covering device in accordance with a further embodiment of the present invention; and

FIG. 11b is a perspective view of a spinal covering device in accordance with a further embodiment of the present invention.

### DETAILED DESCRIPTION OF PARTICULAR EMBODIMENTS

A spinal covering device for covering exposed neural spinal elements after a spinal procedure such as laminectomy, spinal decompression, spinal release, osteotomy and discectomy is described herein. The removal of soft or bony tissues at one or more vertebral levels along the spinal column leaves the neural spinal elements exposed and susceptible to physical trauma. Products such as gelatine foam or fat do not provide a sufficient barrier to prevent potential physical trauma. The spinal covering device advantageously rests on or against remaining soft or bony tissues to minimize the contact between the device and surrounding neural spinal elements by forming a spinal canal portion containing the spinal element. Such reduced contact minimizes adhesion formation between the neural tissues and the device itself.

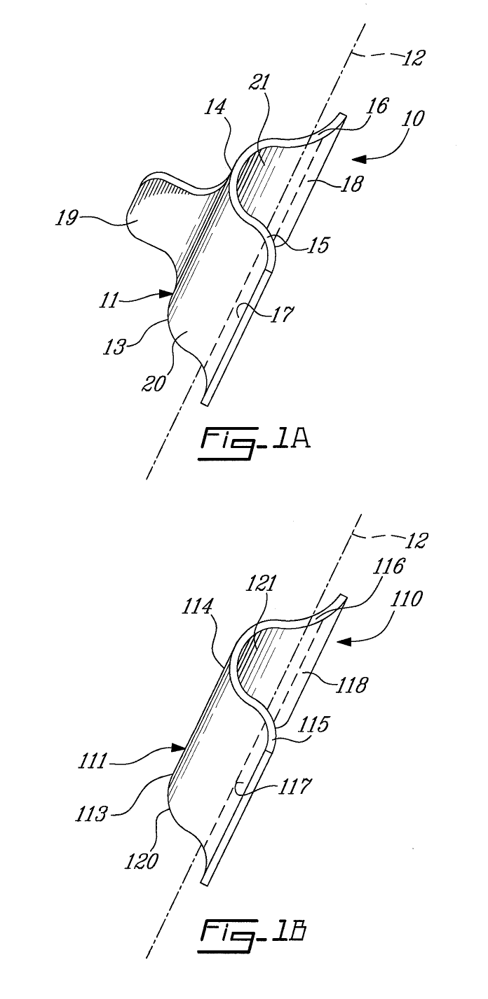

Referring to FIG. 1a, a spinal covering device 10 according to a particular element of the present invention is shown. More particularly, the device 10 comprises a body 11 preferably at least substantially or completely free of openings, and extending longitudinally along an axis 12 from a first end 13 to a second end 14. The body 11 has an arcuate shape defined from a first longitudinal side 15 to a second longitudinal side 16 thereof. The device 10 also comprises a first support plane 17 and a second support plane 18 extending from a respective one of the longitudinal sides 15, 16. The first and second support planes 17, 18 provide a surface area suitable for resting the device 10 against and over remaining soft or bony tissues on opposite sides of the neural element at one or more vertebral levels, such that the device defines a spinal canal and allows protection against compression and injuries. The support planes 17, 18 can alternately be placed such that only end surfaces thereof abut the remaining bony tissues. The device 10 has a member 19, which can be in the form of a spinous process or have any other adequate shape, protruding at least substantially perpendicular from the body 11. In a particular embodiment, the member 19 is used for attachment and stabilisation of paraspinal muscles. The device 10 defines an inner surface 21 formed at least in part by the concave side of the body 11 and defining at least part of an inner surface of the spinal canal, and an outer surface 20 opposite of the inner surface 21, the outer surface 20 allowing bone graft deposition or bone fusion thereon. When the support planes 17, 18 are placed with only end surfaces thereof abutting the remaining bony tissues, the end surfaces also preferably allow bone fusion therewith.

Referring to FIG. 1b, a spinal covering device 110 according to an alternate embodiment of the present invention is shown. More particularly, the device 110 comprises an arcuate shaped body 111 preferably at least substantially or completely free of openings and extending longitudinally from a first end 113 to a second end 114 with the arcuate shape being defined from a first longitudinal side 115 to a second longitudinal side 116. The device 110 also comprises a first support plane 117 and a second support plane 118 extending from the longitudinal sides 115,116 to provide a surface area suitable for resting the device 110 against and over, or alternately along and in abutment with, remaining soft or bony tissues. The device 110 has an inner surface 121 defining at least part of the inner surface of a spinal canal portion and an opposed outer surface 120, and if required, end surfaces, allowing bone graft deposition or bone fusion thereon.

In a particular embodiment, the first and second support planes 17, 18 and 117,118 overlay opposite sides of the remaining vertebral bones.

In a preferred embodiment, the spinal covering device 10,110 is rigid.

The device 10,110 advantageously increases the surface available for bone fusion and for the deposition of bone graft or bone substitute. In a particular embodiment, the device 10,110 promotes or enhances bone growth and healing, such as by being entirely made of or by having the outer surface 20,120 including a bone fusion enhancing or promoting substance, e.g. a bone extender, enhancer or substitute that has osteoconductive, osteoinductive or osteogenic properties. Such substance include, for example, cadaver bone, bone morphogenetic protein, demineralized bone matrix, collagen, suitable polymers, suitable ceramics such as natural coral (calcium carbonate) or coralline hydroxyapatite, or suitable composites such as hydroxyapatite-tricalcium phosphate. Preferably, the device 10,110 becomes incorporated into the remaining bony tissues. More particularly, the remaining bony tissues fuse with the contacting outer surface 20,120 of the device, forming a new spinal canal.

In a particular embodiment the inner surface 21, 121 adjacent the neural spinal elements and defining the inner surface of the spinal canal portion prevent scar formation and bone regrowth. For example the inner surface 21,121 of the device 10,110 can be advantageously impregnated or coated with a drug or substance suitable for preventing scar formation and bone regrowth. As such, the inner surface 21,121 of the device 10,110 may contain an absorptive, saturatable or impregnatable material suitable for acting as a carrier of a drug or substance for preventing scar formation and bone regrowth. Alternately, the device 10,110 may be used in combination with a suitable element or substance preventing scar formation and tissue growth, for example absorbable gelatine, placed between the device 10,110 and the neural spinal element to prevent adhesion.

The actual dimensions of the device 10,110 will vary depending on the particular surgical procedure and on the patient's anatomy. The device is preferably prepared in a size and shape to accommodate a particular spinal procedure. It is further contemplated that the overall length of the device 10,110 can be varied to accommodate spinal procedure involving more than one vertebra. It is also contemplated to provide the device 10,110 in a relatively large dimension for cutting to adequate size upon use.

It is contemplated that the device 10,110 may be prepared from any number of adequate materials known in the art. Preferably, the material is biocompatible. For example, the device can be prepared from an adequate type of ceramic, composite, coral, polymer or porous metal, with the outer surface 20,120 including a bone fusion enhancing or promoting substance, e.g. bone extender, enhancer or substitute that has osteoconductive, osteoinductive or osteogenic properties, as discussed above. In a particular embodiment, the device 10,110 is made of a suitable plastic or ceramic and includes an outer layer of bone morphogenetic protein defining the outer surface 20,120.

The attachment means contemplated for use with the device 10, 110 of the present invention can take any number of forms. In a preferred embodiment, the attachment is to bone. Bone attachment contemplated within the scope of this invention include, but are not limited to, attachment to adjacent spinous processes and lateral attachments such as to lamina facets, transverse processes, articulating processes or the like. It is desirable that no contact is made with the neural elements. Preferably, the attachment means does not extend into the spinal canal or neuroforamen to an extent that would make contact with the dura or nerve roots likely.

In a particular embodiment, the attachment means includes surgical wires, staples, pins, screws or the like. The pins, wire, screws or other attachment means contemplated within this invention are preferably prepared from surgical steel, tungsten, titanium or other suitable materials.

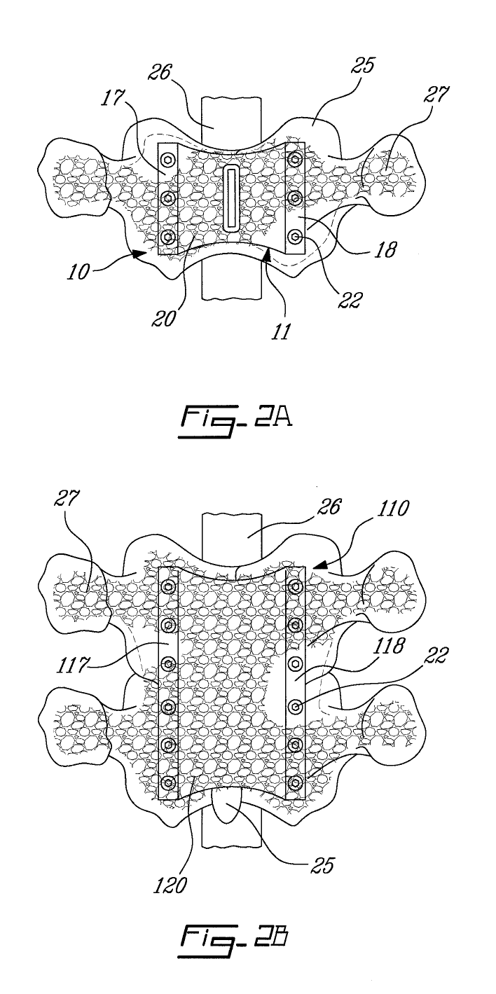

An exemplary application of the device 10,110 is provided in FIGS. 2a and 2b. The device 10, 110 is positioned over the remaining soft or bony tissues and a set of four attachment screws 22 are used to anchor the device 10 to the opposite sides of remaining soft or bony tissues 25 of one vertebra, thus protecting the exposed neural spinal elements 26. Bone graft 27 can be deposited on the outer surface 20,120 of the body 11,111 for bone regrowth or bone fusion with the remaining vertebral bones 25.

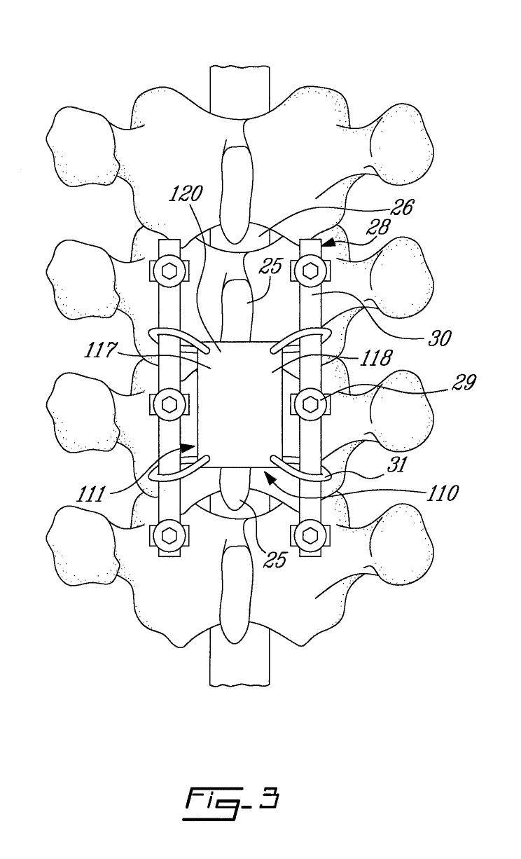

Turning to FIG. 3, a spinal covering device 110 such as shown in FIG. 1b is used with a spinal instrumentation system 28 which is particularly useful where lumbar segmental instability is a problem. As shown in FIG. 3, the device 110 overlays remaining soft or bony tissues 25 and is attached to the spinal instrumentation system 28 which includes pedicle screws 29 and longitudinal rods 30 used to stabilize adjacent vertebras. The spinal covering device 110 overlays one or more vertebral levels and is secured to the rods 30 in a non rigid manner with wires 31. Alternately the spinal covering device 10 of FIG. 1a can also be used with a similar spinal instrumentation system, and both devices 10,110 can be used with alternate types of spinal implants.

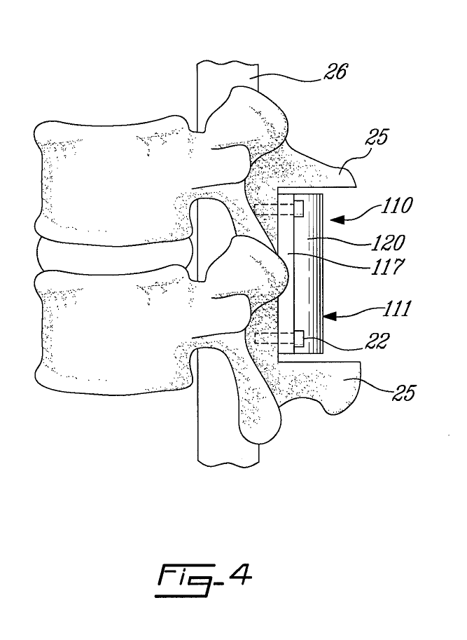

FIG. 4 shows the spinal covering device 110 overlaying the remaining vertebral bones 25 and attached with attachment screws 22. It can be appreciated that the remaining vertebral bones 25 are overlaid by the device 110 over two vertebral levels, similarly to the application of FIG. 3.

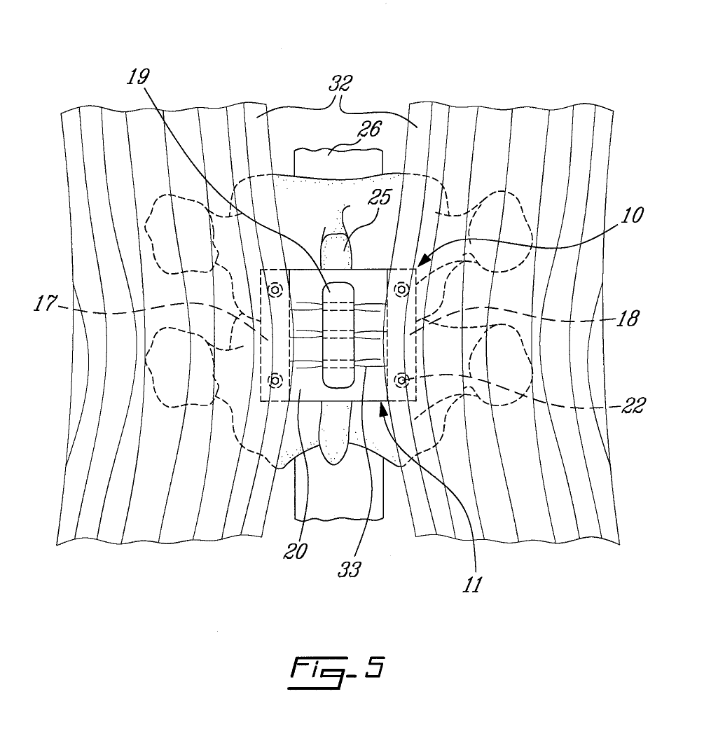

FIG. 5 shows the spinal covering device 10 overlaying the remaining vertebral bones 25 over two vertebral levels and illustrating the attachment of paraspinal muscles 32 to the spinal covering device 10. More specifically, the paraspinal muscles are attached to the member 19 of the device 10, preferably with sutures 33.

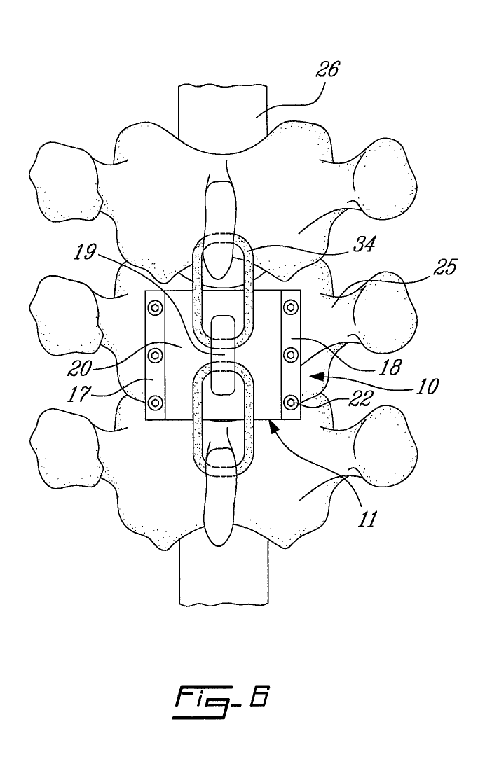

The device 10,110 can also be connected rigidly or non-rigidly to at least one adjacent vertebra to recreate spinal stability. FIG. 6 illustrates an embodiment wherein the device 10 is connected non-rigidly through links 34 to adjacent vertebrae.

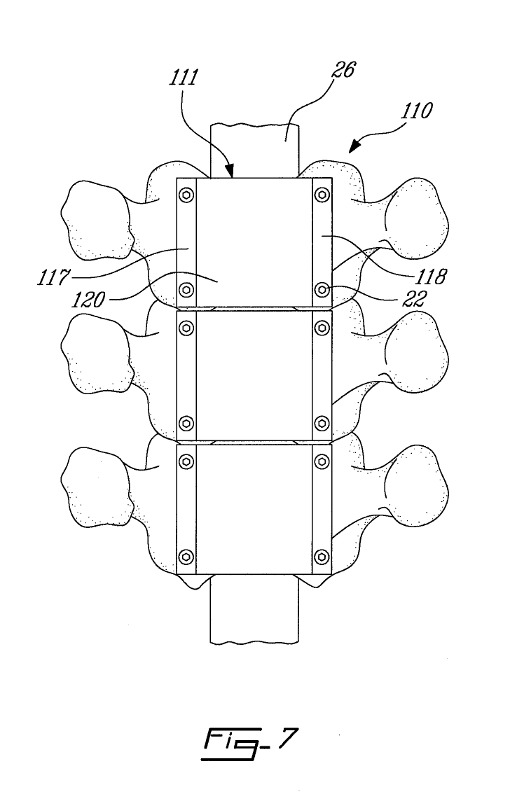

The spinal covering device 10, 110 can also be used to overlay several vertebrae by using a plurality of adjacent single level spinal covering devices such as 110, as shown in FIG. 7.

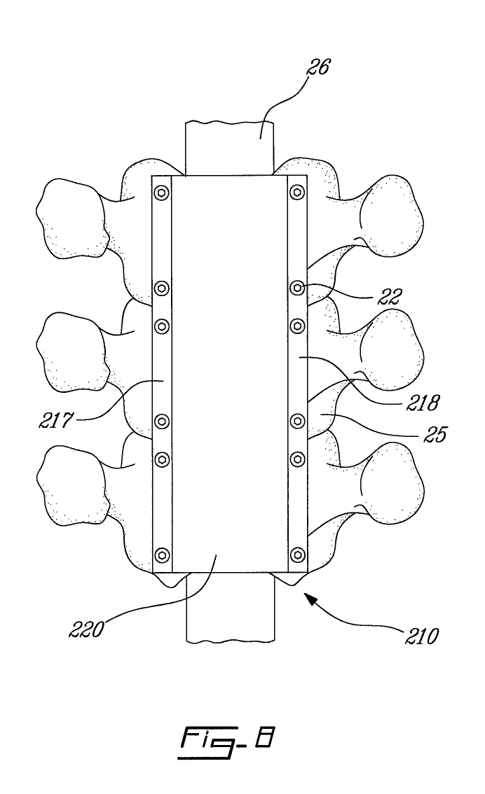

Referring to FIG. 8, a spinal covering device 210 according to another embodiment of the present invention is shown. The device 210 extends to overlay remaining vertebral bones 25 at multiple levels by spanning multiple vertebral segments. Although not shown, the spinal covering device 210 can include one or more members protruding from the outer surface as per the member 19 of the device 10 of FIG. 1a.

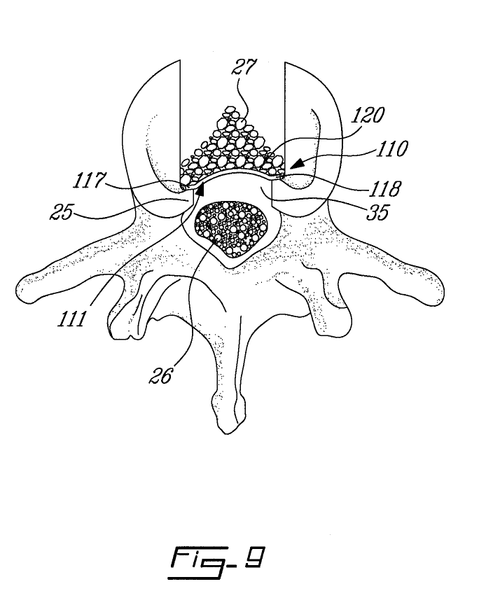

FIG. 9 shows a spinal covering device 110 used on the anterior side instead of the posterior side of the vertebra. More particularly, the first and second support planes 117,118 overlay the remaining vertebral bones 25 of the anterior body on each side of the exposed neural spinal elements 26 and the arcuate shape body 111 forms an expanded spinal canal 35 providing protection against neural compression and injuries. As shown in FIG. 9, bone graft 27 can be deposit on the outer surface 120 of the body 111 for bone regrowth or bone fusion with the remaining vertebral bones 25.

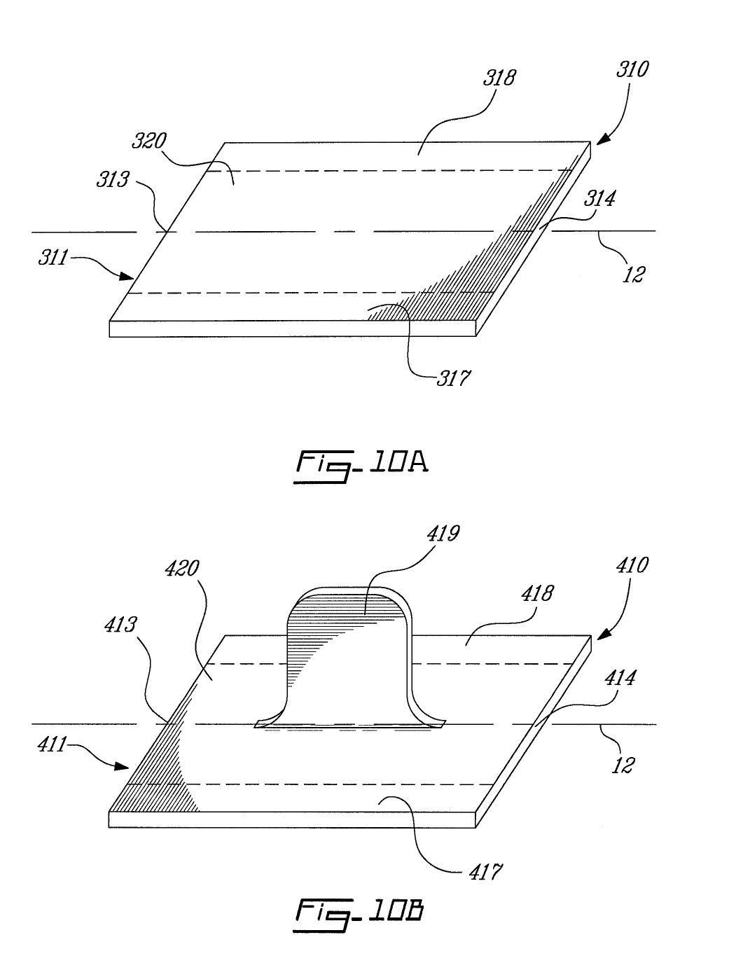

FIG. 10a shows a planar spinal covering device 310 according to an alternate embodiment of the present invention. The device 310 includes a planar body 311 preferably at least substantially or completely free of openings which extends longitudinally along an axis 12 from a first end 313 to a second end 314 for covering exposed neural spinal elements 26 after a spinal procedure. The planar spinal covering device 310 has also a first support plane 317 and a second support plane 318 which are continuous with the planar central portion of the body. The device 310 has an outer surface 320 opposite the side of the device 30 which defines the inner surface of the spinal canal portion, the outer surface 320, and if required, end surfaces, allowing bone graft deposition or bone fusion thereon.

Turning to FIG. 10b, a spinal covering device 410 according to an alternate embodiment is shown. The device 410 is similar to the device 310 and includes a planar body 411, first and second ends 413,414, and opposed first and second support planes 417,418 which are continuous with the planar central portion of the body 411. The device 410 has a member 419 protruding at least substantially perpendicularly from the body 411, which can be used for attachment and stabilisation of paraspinal muscles 32. The device 410 also has an outer surface 420 and if required, end surfaces, allowing bone graft deposition or bone fusion thereon.

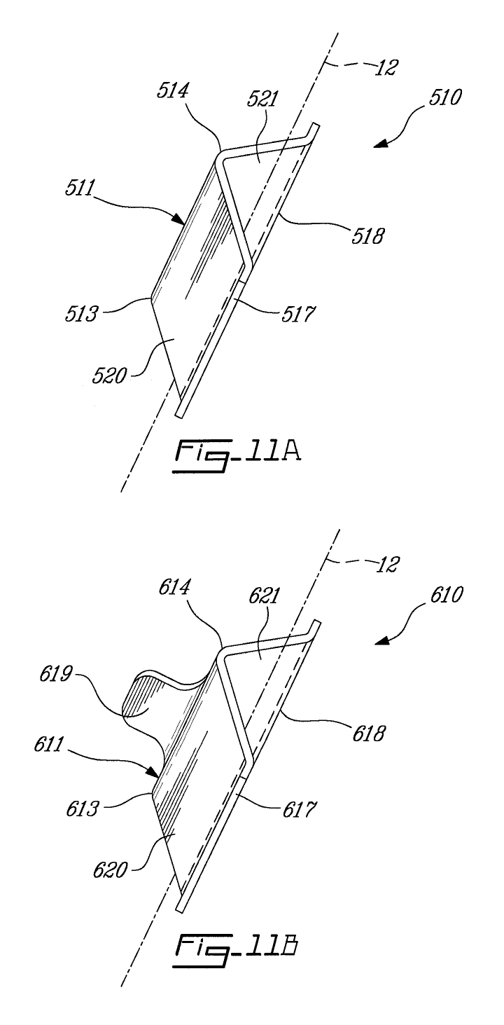

FIG. 11a shows a triangular spinal covering device 510 according to an alternate embodiment of the present invention. More particularly, the device 510 includes a triangular shaped body 511 preferably at least substantially or completely free of openings and extending longitudinally from a first end 513 to a second end 514 for covering exposed neural spinal elements 26 after a spinal procedure. The triangular spinal covering device 510 also has a first support plane 517 and a second support plane 518 extending from a respective one of the longitudinal sides of the body 511. The device 510 has an inner surface 510 forming an inner surface of the spinal canal portion, and an outer surface 520 and if required, end surfaces, allowing bone graft deposition or bone fusion.

Turning to FIG. 11b, a spinal covering device 610 according to an alternate embodiment is shown. The device 610 is similar to the device 510 and includes a triangular body 611, first and second ends 613,614, a first support plane 617 and a second support plane 618. The device 610 further includes a member 619 protruding at least substantially perpendicularly from the body 611. The device 610 also has an inner surface 621, and an outer surface 620 and if required, end surfaces, allowing bone graft deposition or bone fusion thereon.

All of the spinal covering devices 210, 310, 410, 510 and 610 can include an inner surface preventing scar formation and bone regrowth and be used in any appropriate one of the applications shown and described and variants thereof.

1. A spinal covering device for covering exposed neural spinal elements after a spinal procedure, the device comprising a body at least substantially free of openings and extending longitudinally from a first end to a second end, and first and second support planes extending longitudinally along the body, the first and second support planes being adapted to overlay or abut remaining tissues on opposite sides of the exposed neural spinal elements such that the device forms a spinal canal portion protecting the neural spinal elements, the device having an inner surface at least partially defining an inner surface of the spinal canal portion and an outer surface opposite of the inner surface, at least the outer surface allowing bone graft deposition or bone fusion thereon.

2. The spinal covering device according to claim 1, wherein the body has an arcuate shape, a triangular shape or a planar shape.

3. The spinal covering device according to claim 1, wherein the inner surface includes a substance preventing scar formation and bone regrowth.

4. The spinal covering device according to claim 1, wherein the outer surface includes a bone extender, enhancer or substitute that has osteoconductive, osteoinductive or osteogenic properties.

5. The spinal covering device according to claim 4, wherein end surfaces of the support planes also include a bone extender, enhancer or substitute that has osteoconductive, osteoinductive or osteogenic properties.

6. The spinal covering device according to claim 1, further comprising a member protruding at least substantially perpendicularly from the outer surface.

7. The spinal covering device according to claim 1, wherein at least the outer surface of the device includes one of cadaver bone, bone morphogenetic protein, demineralized bone matrix, collagen, natural coral, coralline hydroxyapatite, and hydroxyapatite-tricalcium phosphate.

8. A spinal covering device for covering at least one exposed neural spinal element after a spinal procedure comprising:
an arcuate shaped body at least substantially free of openings, the arcuate shape being defined from a first longitudinal side to a second longitudinal side of the body; and
first and second support planes respectively extending from the first and second longitudinal sides, the first and second support planes being adapted to overlay or abut remaining vertebral bones such that the device extends over and spaced apart from the exposed neural spinal elements to form a spinal canal portion;
the device having an inner surface at least partially defining an inner surface of the spinal canal portion and an outer surface opposite of the inner surface, at least the outer surface including a bone extender, enhancer or substitute that has osteoconductive, osteoinductive or osteogenic properties.

9. The spinal covering device according to claim 8, further comprising a member protruding at least substantially perpendicularly from the outer surface.

10. The spinal covering device according to claim 8, wherein the inner surface includes a substance preventing scar formation and bone regrowth.

11. The spinal covering device according to claim 8, wherein end surfaces of the support planes also include a bone extender, enhancer or substitute that has osteoconductive, osteoinductive or osteogenic properties.

12. The spinal covering device according to claim 8, wherein at least the outer surface of the device includes one of cadaver bone, bone morphogenetic protein, demineralized bone matrix, collagen, natural coral, coralline hydroxyapatite, and hydroxyapatite-tricalcium phosphate.

13. A method for protecting at least one exposed neural spinal element following a spinal procedure, comprising:
positioning a spinal covering device on remaining soft or bony tissues on each side of the at least one exposed neural spinal element or in abutment with the remaining bony tissues to extend over and spaced apart from the at least one exposed neural spinal element; and
contacting bone graft or bone substitute material added over or included in an outer surface of the spinal covering device with the remaining bony tissues to allow the bone graft or bone substitute material to fuse with the remaining bony tissues.

14. The method of claim 13, further comprising adding the bone graft or bone substitute material over the outer surface after positioning the spinal covering device.

15. The method of claim 13, wherein the spinal covering device is a first device, the method further comprising positioning at least a second spinal covering device adjacent the first device.

16. The method of claim 13, further comprising attaching the device to a spinal instrumentation system attached to at least adjacent vertebra.

17. The method of claim 13, further comprising attaching the device to adjacent vertebrae to recreate spinal stability.

18. The method of claim 17, wherein the device is attached rigidly to the adjacent vertebrae.

19. The method of claim 13, further comprising positioning the device to span multiple vertebral segments.

20. The method of claim 13, further comprising attaching a member protruding from the outer surface of the device to paraspinal muscles.

21. The method of claim 13, further comprising rigidly attaching the device to the remaining soft or bony tissues.

22. The method of claim 21, wherein the device is attached to the soft or bony tissues through at least one of surgical wires, staples, pins and screws.
